(19) United States
(12) Patent Application Publication
(10) Pub. No.: US 2012/0191029 A1
(43) Pub. Date: Jul. 26, 2012

(54) CONNECTION SYSTEM FOR FLUID CONNECTION IN MEDICINE

(76) Inventors: Hans Juergen Hopf, Zirndorf (DE); Norbert Kassai, Oberasbach (DE); Alexander Hopf, Nuernberg (DE); Michael Hopf, Zirndorf (DE)

(21) Appl. No.: 13/387,967

(22) PCT Filed: Jun. 22, 2010
(86) PCT No.: PCT/EP10/58828

§ 371 Date: Apr. 12, 2012

(30) Foreign Application Priority Data
Jul. 28, 2009 (DE) 20 2009 005 077.7
Oct. 23, 2009 (DE) 10 2009 044 319.3
Oct. 23, 2009 (DE) 20 2009 013 408.3
Jan. 22, 2010 (DE) 20 2010 000 078.5

(51) Int. Cl. A61M 25/16; F16L 37/10; A61M 1/02; F16L 37/08

(52) U.S. Cl. 604/7; 285/305; 604/535

(57) ABSTRACT

The present invention relates to a lockable connection system for components in medicine and medical technology that have fluids passing through them with a female, tube- or cone-shaped receptacle (11, 21, 41, 51) having an interior receptacle section and an external first fixing section, and a male, tube- or cone-shaped insertion element (12, 62, 72, 82, 92, 112, 132, 152) to be accommodated in the female receptacle section (11, 21, 41, 51) and an external second fixing section (14, 60, 70, 80, 90, 100, 110, 120, 130, 140, 150) interacting with the first fixing section for fixing the connection system. The invention is characterized in that the first or second fixing section has at least two radially outward-protruding lobes (13, 13′, 23, 23′, 33, 33′, 43, 43′, 53, 53′), the radial external edge of which lies on a diameter greater than 7.83 mm and/or whose axial thickness at the base is greater than 1.3 mm. Alternatively, the invention is characterized in that the first or second fixing section has at least three radially outward-protruding lobes.

The present invention relates to a connection system for fluid connection as used in particular in medicine and medical technology.

Connection systems for fluid connections are known in the prior art and are used in particular in medicine and medical technology. Here the connections are used more particularly in the field of infusions, artificial feeding, syringes and injection kits in transfusions and, more particularly, for the supply and transfer of various fluid media and as so-called injection equipment for medical and pharmaceutical equipment, which comprises several components.

In medicine absolute safety when using syringes, infusion tubes, cannulas etc. is of the utmost priority. Therefore, in medical technical the Luer or Luer lock principle has prevailed over the previously customary connection systems.

Luer lock is a standardised connection system for cannulas, syringes and infusion tubes in medical fields.

Sealing is achieved by means of a conical design of the connection parts, known as the Luer cone. The inner cone on one side of the connection is known as “female” and the outer cone on the opposite side as “male”. The cone has a slope of 6%.

If the cone is provide with a thread and union unit to secure/lock the connection against accidental loosening, the system is known as Luer lock. The simpler version without a screw thread on the syringe side is known as a Luer connection, Luer plug or also Luer slip.

The Luer lock system guarantee compatibility between different manufacturers and is internationally recognised. The Luer lock connection for the reversible connection of syringes, cannulas, infusion tubes, spinal needles and suchlike has gained acceptance throughout the world. This is of great advantage, particularly when used in the context of internal aid and disaster relief.

At the same time the universality of the system also has a dramatic drawback and risk associated therewith. The Luer lock connection conceal dangerous possibilities of confusion, as it is compatible for vascular (venous and arterial), and spinal/epidural (spinal marrow) application, partly also for enteral (e.g. nutritional systems and applications) and respiratory (airway) applications. Essentially the connection method does not therefore prevent, for example, a drug for intravenous administration being given incorrectly (into the spinal marrow fluid). Unfortunately there have been such cases of confusion with a fatal outcome.

In addition the connections system known in the prior art that have fluids flowing through them also have the disadvantage that they are, amongst other things, made of plastic, during the processing of which softening agents, such as bisphenol A, for example, are used. When using the fluids system through which fluids flow it cannot be ruled out that particles of the softening agents are given off, whereby it is known that these softening agent materials can harm human health. Among other things, these softening agents are ascribed effects on the human hormone balance and carcinogenic effects.

A further disadvantage of the fluid connections known in the prior art is that in use they are used more or less openly, so that particularly when no fluid is being administered to the patient they come into contact with the air in the treatment rooms even with other objects and persons can thereby become contaminated for example. In certain circumstances this contamination can lead to severe infections in the persons to be treated.

The aim of the present invention is to at least partially overcome the disadvantages known in the prior art, more particularly the risk to the possibility of confusion, the possible risk of exposure of the patient to undesirable substances and the risk of infection of the patient.

The above aim is achieved by way of the connection system in accordance with the invention through claim 1, as well as the male and female connection components of the connection system in accordance with the invention and through the use of the connection system in medicine and medical technology, and through the use of appropriate materials for the manufacture of at least the surface coming into contact with the fluid. Preferred forms of embodiment of the connection system as well as the corresponding male and female connection components are the subject matter of the corresponding sub-claims.

The lockable connection system in accordance with the invention for components through which fluids flow for use in medicine and medical technology, comprises female tube or cone-shaped receptacle with an interior receptacle section and an external first fixing section, as well as male, tube or cone-shaped insertion element for holding in the female receptacle section with an external second fixing sections which interacts with the first fixing section in order to fix the connection system. The lockable connection system in accordance with the invention is characterised in that the first or second fixing section has at least two outwardly protruding lobes, the radial outer edge of which lies on a diameter of greater than 7.83 mm and/or the axial thickness of which at the base is greater than 1.3 mm.

Taken to mean fixing section in the sense of the present invention are devices for securing or locking the connection.

The radial outer edge in accordance with the present invention is the outer diameter of the lockable inner cone including extension through the lobes.

In accordance with a further, particularly preferred form of embodiment of the lockable connection system in accordance with the invention the outer edge lies on a diameter which is greater than 7.83 mm and less than 20 mm, more particularly greater than 8 mm and less than 10 mm and in particular is around 9 mm, and/or the axial thickness is greater than 1.3 mm and less than 5 mm, more particularly greater than 1.5 mm and less than 3 mm, and more particularly around 1.8 mm to 2.5 mm.

In accordance with further, particularly preferred form of embodiment of the lockable connection system in accordance with the invention for components through which fluid flow for use in medicine and medical technology the system has a female tube or cone-like receptacle with an internal receptacle section and an external first fixing section, as well as a male tube or cone-like insertion element for holding in the female receptacle section with an external second fixing section which interacts with the first fixing section in order to fix the connection system. The lockable connection system in accordance with the invention is characterised in that the first or second fixing section has at least three radially outwardly protruding lobes.

In accordance with a further, particularly preferred form of embodiment of the connection system in accordance with the invention the second or first fixing section has a sleeve for accommodating the lobe, which has an internal thread, more particularly a multiple thread internal thread or a bayonet fitting. In accordance with a further preferred form of embodiment of the connection system the number of threads of the internal thread of the sleeve for accommodating the lobes of the second or first fixing section corresponds with the number of lobes on the corresponding first of second fixing section.

In accordance with a further particularly preferred form of embodiment of the connection system in accordance with the invention the sleeve of the second or first fixing section is designed for holding the first or second fixing section so that it can be moved, more particularly turned, independently of the male insertion element and/or the female receiving element.

In accordance with a further, preferred form of embodiment of the connection system in accordance with the invention the internal thread of the sleeve for holding the lobes of the second or first fixing section is designed as a self-locking thread.

In accordance with further preferred form of embodiment of the connection system in accordance with the invention the male insertion element has a cone whereby this cone has two different slope angles α, β and, more particularly, the slope β of the cone in the area of the base of the insertion element is greater than the slope β of the cone at the end area of the insertion element and this greater slop α is in particular between 65° and 85°, preferably between 70° and 80° and particularly preferably around 78°.

In accordance with a further particularly preferred form of embodiment of the connection system in accordance with the invention the male insertion element has a cone, where at its base his cone has an external diameter of between 5.8 mm and 6.4 mm, preferably between 6.0 and 6.2 mm, particularly preferably 6.1 mm.

In accordance with a further preferred form of embodiment of the lockable connection system in accordance with the invention it is characterised in that the components through which fluids flow are in particular infusion or transfusion tubes, multi-way valves, multiple distributors, injection equipment such as needles, accesses, syringes, enteral feeding systems and applications or suchlike and combinations thereof.

In accordance with a further preferred form of embodiment the connection system in accordance with the invention is characterised in that at least the surfaces of the connection system coming into contact with the fluid, more particularly the internal surfaces of the connection system, are at least in sections made of a material which has antiseptic and/or antimicrobial properties.

Such antimicrobial or antiseptic materials are, for example, materials such as highly porous silver, ion-free silver, silver compounds and, more particularly, microsilver, compounds releasing compounds, combinations thereof and suchlike. Preferably these are arranged in appropriate surface, whereby in accordance with a further particularly preferred form of embodiment the relevant materials are also incorporated in the plastic of which the connection system and/or its components are made. Thus, more particularly, a highly porous silver, which is preferably essentially ion-free, can be mixed into the plastic, of which the components of the connection system can at least in parts be made. Alternatively, coating the surfaces with a relevant material and/or material combination is also within the scope of the present invention.

In accordance with a further specially preferred form of embodiment of the present invention an amorphic copolyester is used for the production of a lockable connection system for components through which fluids flow for medicine and medical technology, such as, in particular, infusion or transfusion tubes, multi-way valves, multiple distributors, injection equipment such as needles, accesses or suchlike and combinations thereof, whereby the connection system at least has a female, tube-like receptacle which has an internal receptacle section and an external first fixing section and/or a male tube-shaped insertion element for accommodating in the female receptacle section and an external second fixing section which interacts with the first fixing section to fix the connection system. The system is characterised in that at least in sections the surfaces of the connection system coming into contact with the fluid are made of an amorphic copolyester.

Furthermore, in addition to the copolyester, other materials are used to produce the connection system in accordance with the invention which are selected from a group containing duroplastic and thermoplastic synthetic materials, more particularly polylphenyl sulphide, polypropylene, poly-1-butene, polyvinylchloride, polyvinylidene chloride, polymethyl metaacrylate, polyacryl nitrile, polystyrene, polysulphone, polyacetal, polyvinyl alcohol, polyvinyl acetate, ionomers, fluoroplastics, polyethylene, polyamide, more particularly a partial aromatic polyamide, polycarbonate, polyester, copolyester, polyphenyl oxide, polysulphone, polyvinyl acetate, polyurethane and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, polyvinyl chloride, unsaturated polyester resin, antimicrobial or antiseptic materials, for example highly porous silver, ion-free silver, silver compounds and in particular micro-silver, metal ion-releasing compounds as well as combinations thereof and suchlike.

In accordance with a further particularly preferred form of embodiment at least partially materials are used for the production of the connection system in accordance with the invention which do not contain any softening agents, more particularly no phthalates and no bisphenol A.

The aim of the invention is also achieved through a tube- or cone-shaped receptacle or an insertion element for a lockable connection system which has an external fixing section which has at least two outwardly protruding lobes, the radial external edge of which lies on a diameter which is greater than 7.83 mm and/or the axial thickness of which at the base is greater than 1.3 mm or which has at least there outwardly protruding lobes.

In accordance with a further preferred form of embodiment of the pipe or cone-shaped receptacle or the insertion element in accordance with the invention for a lockable connection system, these have an external fixing section with a sleeve for accommodating the lobes, the radial external edge of which lies on a diameter which is greater than 7.83 mm and/or the axial thickness of which at the base is greater than 1.3 mm and/or there are at least three radially outwardly protruding lobes. Moreover, the sleeve is characterised in that it has an internal thread, more particularly a multiple-thread internal thread or a bayonet fitting.

In accordance with a further particularly preferred form of embodiment of the tube or cone-shaped receptacle or the insertion element in accordance with the invention for a lockable connection system the sleeve is designed so that is can be move, more particularly turned, independently of the male insertion element and the female receptacle section.

In accordance with further preferred form of embodiment of the tune or cone-like receptacle or the insertion element in accordance with the invention for a lockable connection system the male insertion element has a cone whereby this cone has two different slope angles α, β and, more particularly, the slope α of the cone in the area of the base of the insertion element is greater than the slope β of the cone at the end area of the insertion element and this greater slop β is in particular between 65° and 85°, preferably between 70° and 80° and particularly preferably around 78°.

In accordance with further preferred form of embodiment of the tune or cone-like receptacle or the insertion element in accordance with the invention for a lockable connection system the male insertion element has a cone, where at its base his cone has an external diameter of between 5.8 mm and 6.4 mm, preferably between 6.0 and 6.2 mm, particularly preferably 6.1 mm.

The present invention also comprises a device consisting of a tube or cone-shape receptacle and an insertion element in accordance with the invention, characterised in that at least in parts the surfaces of the components, through which fluid flows, coming into contact with the fluid, more particularly the internal surfaces of the connection system, are at least in sections made of an antiseptic and/or antimicrobial material. Such antimicrobial or antiseptic materials are, for example, materials such as highly porous silver, ion-free silver, silver compounds and, more particularly, microsilver, compounds releasing compounds, combinations thereof and suchlike. Preferably these are arranged in appropriate surfaces, whereby in accordance with a further particularly preferred form of embodiment the relevant materials are also incorporated in the plastic of which the connection system and/or its components are made. Thus, more particularly, a highly porous silver, which is preferably essentially ion-free, can be mixed into the plastic, of which the components of the connection system can at least in parts be made. Alternatively, coating the surfaces with a relevant material and/or material combination is also within the scope of the present invention.

In accordance with a further specially preferred form of embodiment of the device consisting of a tube or cone-shape receptacle and an insertion element in accordance with the invention an amorphic copolyester is used for the production of a lockable connection system for components through which fluids flow for medicine and medical technology, such as, in particular, infusion or transfusion tubes, multi-way valves, multiple distributors, injection equipment such as needles, accesses or suchlike and combinations thereof, whereby the connection system at least has a female, tube-like receptacle which has an internal receptacle section and an external first fixing section and/or a male tube-shaped insertion element for accommodating in the female receptacle section and an external second fixing section which interacts with the first fixing section to fix the connection system. The system is characterised in that at least in sections the surfaces of the connection system coming into contact with the fluid are made of an amorphic copolyester

Furthermore, in addition to the copolyester, other materials are used to produce the connection system in accordance with the invention which are selected from a group containing duroplastic and thermoplastic synthetic materials, more particularly polylphenyl sulphide, polypropylene, poly-1-butene, polyvinylchloride, polyvinylidene chloride, polymethyl metaacrylate, polyacryl nitrile, polystyrene, polysulphone, polyacetal, polyvinyl alcohol, polyvinyl acetate, ionomers, fluoroplastics, polyethylene, polyamide, more particularly a partial aromatic polyamide, polycarbonate, polyester, copolyester, polyphenyl oxide, polysulphone, polyvinyl acetate, polyurethane and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, polyvinyl chloride, unsaturated polyester resin, antimicrobial or antiseptic materials, for example highly porous silver, ion-free silver, silver compounds and in particular micro-silver, metal ion-releasing compounds as well as combinations thereof and suchlike.

In accordance with a further particularly preferred form of embodiment at least partially materials are used for the production of the connection system in accordance with the invention which do not contain any softening agents, more particularly no phthalate and no bisphenol A.

In accordance with a further preferred form of embodiment of the connection system in accordance with the invention at least parts of the connection system are made of a material selected from a group containing duroplastic and thermoplastic synthetic materials, more particularly polylphenyl sulphide, polypropylene, poly-1-butene, polyvinylchloride, polyvinylidene chloride, polymethyl metaacrylate, polyacryl nitrile, polystyrene, polysulphone, polyacetal, polyvinyl alcohol, polyvinyl acetate, ionomers, fluoroplastics, polyethylene, polyamide, more particularly a partial aromatic polyamide, polycarbonate, polyester, copolyester, polyphenyl oxide, polysulphone, polyvinyl acetate, polyurethane and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, polyvinyl chloride, unsaturated polyester resin, antimicrobial or antiseptic materials, for example highly porous silver, ion-free silver, silver compounds and in particular micro-silver, metal ion-releasing compounds as well as combinations thereof and suchlike.

The present invention also includes the use of the lockable connection system in accordance with the invention in medicine and in medical technology, more particularly for the supply and transfer of various flow media, more particularly for gravity infusion, for pump transfer systems, tube feeding systems, enteral feeding systems and applications, injections as well as combination thereof and suchlike.

In accordance with a further specially preferred form of embodiment the lockable connection system in accordance with the invention is used in a Y-connector. Here, at least one connection point of the Y-connector, for a catheter for example, has at least one part of the lockable connection system in accordance with the invention, i.e. a female tube or cone-shaped receptacle which has an internal receptacle section and an external first fixing section or a male tube or cone-shaped insertion element for accommodating in a female receptacle section and an external second fixing section which interacts with the first fixing section for fixing the connection system.

Y-connectors in the sense of the present invention is taken to mean connection elements which are largely Y-shaped accordingly have on connection point on one side and at least two connections on the other side.

In accordance with a further particularly preferred form of embodiment the lockable connection system in accordance with the invention is used on multi-way valve. Here, at least one connection point of the multi-way valve has one part of the lockable connection system in accordance with the invention, i.e. a female tube or cone-shaped receptacle which has an internal receptacle section and an external first fixing section or a male tube or cone-shaped insertion element for accommodating in a female receptacle section and an external second fixing section which interacts with the first fixing section for fixing the connection system.

Multi-way valve in the sense of the present invention is taken to mean taken to mean lockable connection elements in which the flow between the available connection points can be controlled by an element, the valve, arranged between them.

In accordance with a further particularly preferred form of embodiment the lockable connection system in accordance with the invention is used on a closing cap. Here the closing cap has one part of the connection system in accordance with the invention, i.e. a female tube or cone-shaped receptacle which has an internal receptacle section and an external first fixing section or a male tube or cone-shaped insertion element for accommodating in a female receptacle section and an external second fixing section which interacts with the first fixing section for fixing the connection system.

In accordance with a further, particularly preferred form of embodiment of the lockable connection system in accordance with the invention on a closing cap a lug is attached thereto, whereby the lug is more particularly connected to the closing cap by way of an at least partially elastic elongated section.

The closing cap can be connected to the relevant connection partner of the closing cap by means of the lug.

In accordance with a further particularly preferred form of embodiment of the lockable connection system in accordance with the invention on a closing cap a lug is attached to the closing cap, whereby this attachment is by way of a further lug. The closing cap can have a receptacle for accommodating the lug. The two lugs can also be connected to each other by way of an at least partially elastic elongated section.

The invention will be described below with the aid of a preferred example of embodiment, whereby it should be pointed out that example also covers derivations and/or additions which are directly evident to a person skilled in the art. In addition, this preferred example of embodiment does not restrict the invention so that derivations and additions are included in the scope of the invention.

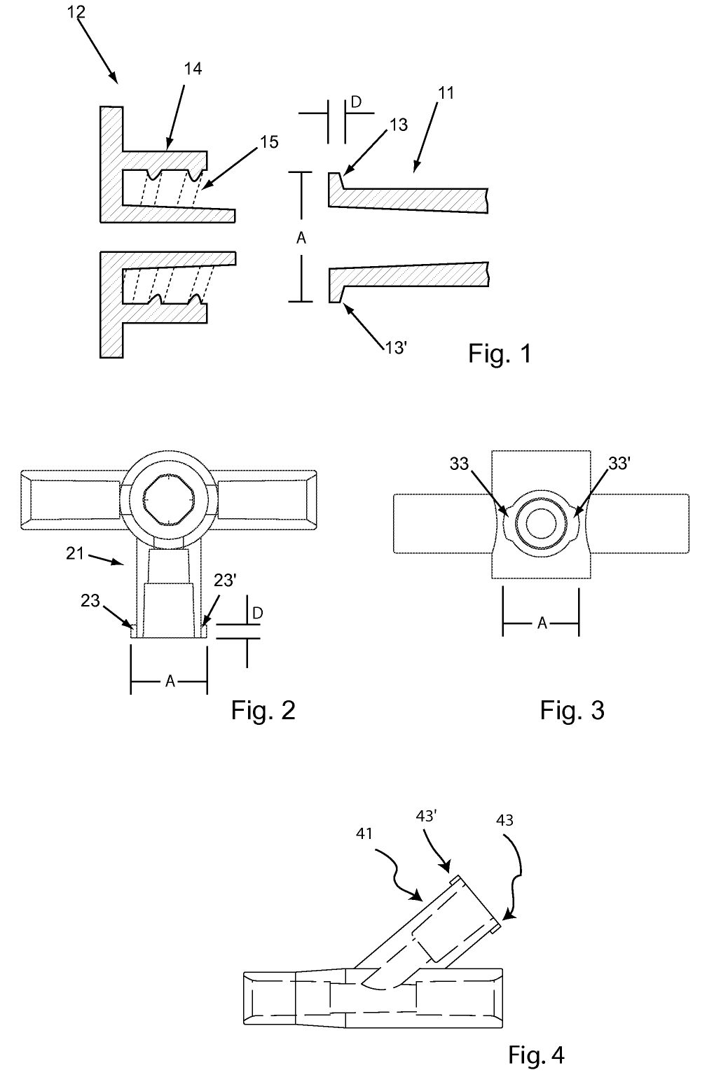

FIG. 1 shows a schematic view of the connection system in accordance with the invention;

FIG. 2 shows a view from above of a multi-way valve with a lockable internal cone in accordance with the invention;

FIG. 3 shows a front view of a lockable internal cone in accordance with the invention;

FIG. 4 shows a side view of a Y connector with a lockable internal cone in accordance with the invention;

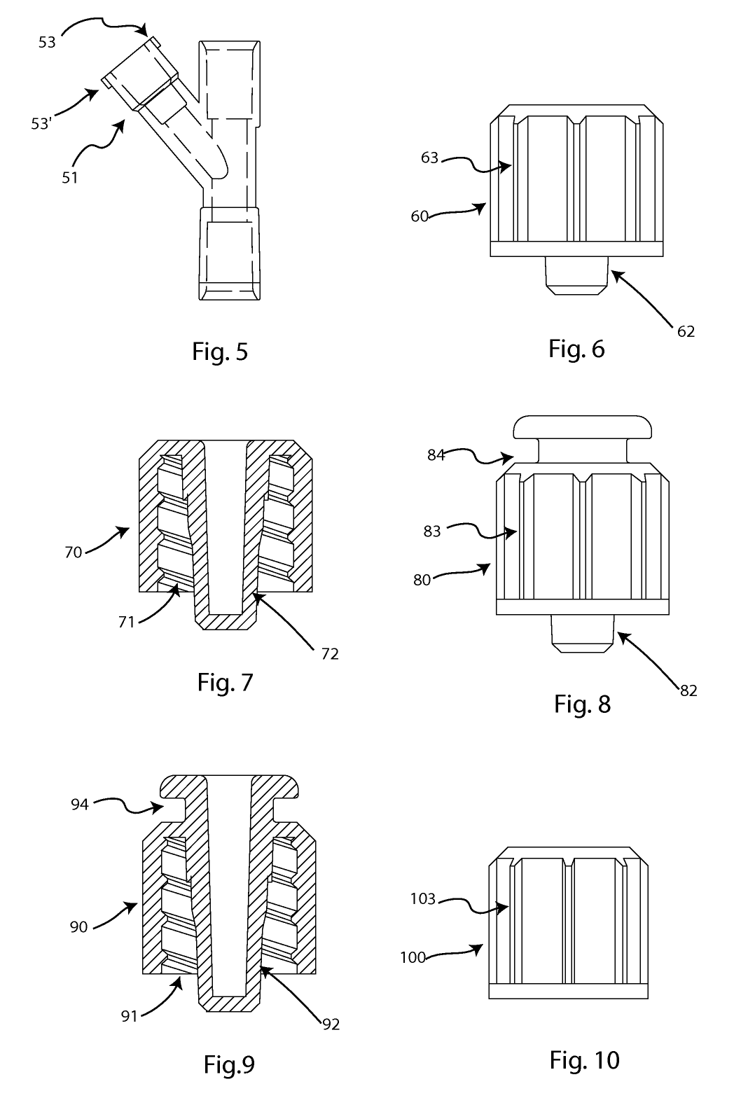

FIG. 5 shows a side view of a Y connector with a lockable internal cone in accordance with the invention;

FIG. 6 shows a side view of a closing cap with a lockable external cone in accordance with the invention;

FIG. 7 shows a cross-section of a lockable external cone in accordance with the invention;

FIG. 8 shows a side view of a closing cap with a receptacle area for a lug with a lockable external cone in accordance with the invention;

FIG. 9 shows a cross-section of a closing cap with a receptacle area for a lug with a lockable external cone in accordance with the invention;

FIG. 10 shows a side view of a closing cap with a lockable external cone in accordance with the invention whereby the cone is shorter than the fixing section;

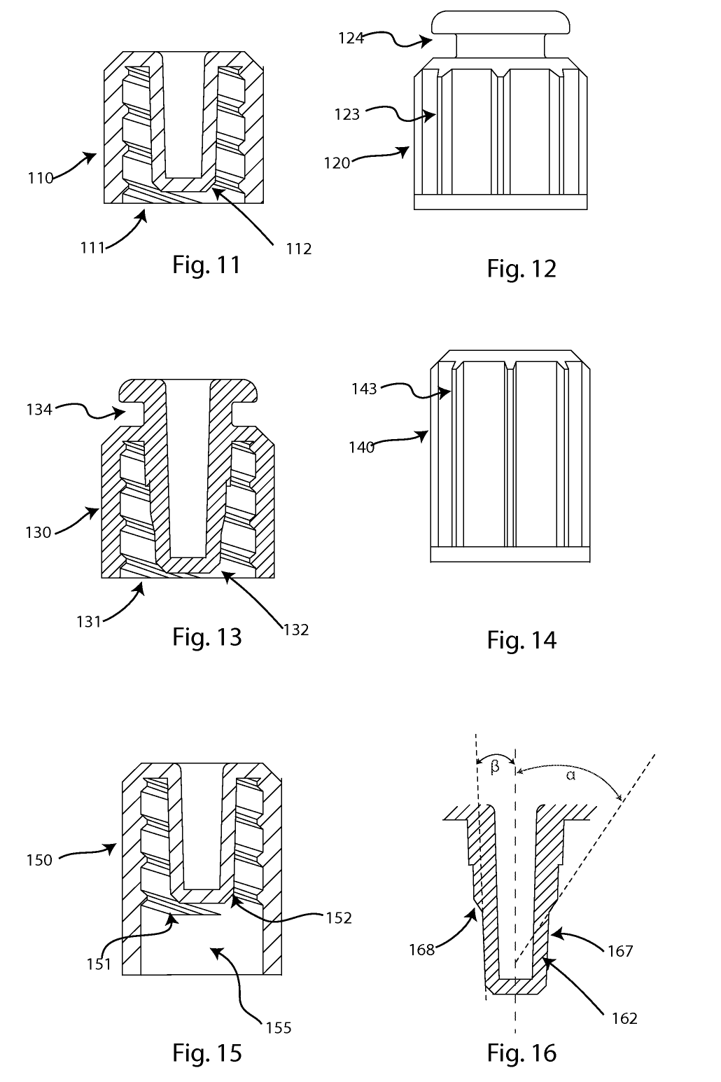

FIG. 11 shows a cross-section of a closing cap with a lockable external cone in accordance with the invention whereby the cone is shorter than the fixing section;

FIG. 12 shows a side view of a closing cap with a receptacle area for a lug with a lockable external cone in accordance with the invention whereby the cone is shorter than the fixing section;

FIG. 13 shows a cross section of a closing cap with a receptacle area for a lug with a lockable external cone in accordance with the invention whereby the cone is shorter than the fixing section;

FIG. 14 shows a side view of a closing cap with a lockable external cone whereby the cone is shorter than the fixing section;

FIG. 15 shows a cross-section of a closing cap with a lockable external cone whereby the cone is shorter than the fixing section;

FIG. 16 shows a male insertion element with two slope angles;

The example of embodiment in accordance with FIG. 1 shows a lockable connection system in accordance with the invention comprising a female 11 and a male 12 connection element with corresponding fixing sections. The female connection element 11 is a lockable internal cone which on the outside has a fixing section in the form of two lobes 13, 13′. The internal cone has a slope of 6%. Section A denotes the radial outer edge, D the axial thickness of the lobes. The male connection element 12 is a lockable external cone. The slope of the cone is 6%. The male connection element has a fixing section 14 which is designed as a sleeve with an internal thread. The conical connection elements, comprising the internal cone and the external cone, are in accordance with the Luer standard for connection systems. Through aligned variation of the fixing sections of the female and the male connection elements matching fixing sections, which rule out confusion, are produced, in a similar manner to the key and lock principle. If the fixing section designed as a sleeve with an internal thread is movable applied to the corresponding connection element this allow unfixed (unlocked) connection of the component through which fluid flow based on the Luer standard. Through firm, i.e. immobile arrangement of the fixing elements this can be prevented. By way of such a design the connection of connection element with different fixing elements can be avoided.

It is also the subject matter of the invention that the sleeve of the fixing section is arranged to be movable to the male insertion element or to the female receptacle section, more particularly that independently of the male insertion element or the female receptacle section is rotatable about these. Through this arrangement of the sleeve the fixing sections can be firmly connected to each other without the male insertion element or the female receptacle section having to be moved and/or their position changed. This arrangement of the sleeve is particularly advantageous if two components are to be connected to the connection system which themselves cannot, or should not, be moved or have to be connected to each other in a certain position.

A variation of the fixing element in accordance with the present invention can relate both to the number and the shape of the lobes (shown in FIG. 2 and FIG. 3). These changes imply a simultaneous change of the corresponding fixing section, designed in the form of a sleeve with an internal thread or bayonet fitting. This can on the one hand related to the number of threads, and also the form of the thread. Naturally the simultaneous change in the number and shape of the lobes, with corresponding adaptation of corresponding fixing section designed as a sleeve is a variation in accordance with the present invention.

FIG. 2 shows a lockable internal cone 21 of a lockable connection system in accordance with the invention in a form of embodiment on a multi-way valve. The external fixing section in this form of embodiment is characterised in that it has two radially outwardly protruding lobes 23, 23′. The radial outside edge A is here on a diameter greater than 7.83 mm and/or the axial thickness D on the base of the lobes is more than 1.3 mm.

FIG. 3 shows a front view of a lockable connection system in accordance with the invention in a form of embodiment on a multi-way valve in accordance with FIG. 2. The external fixing section in this form of embodiment is characterised in that it has two radially outwardly protruding lobes 23, 23′. The radial outside edge A is here on a diameter greater than 7.83 mm.

In a further example of embodiment, shown in sections in FIG. 16, the male insertion element is designed as a cone 162 whereby this cone 162 has at least two different slope angles α, β. While the slope angle β at the cone end 167 essentially has a slope of 6%, the slope angle α of the cone is greater at is base in an area between the cone end and the base 168. In accordance with the invention the slope of the cone on the section towards the base of the cone 168 is between 65° and 85°, preferably between 70° and 80° and particularly preferably around 78°. The outer diameter of the cone of the male insertion element at the end of the section with the greater slope 168 in the direction of the base of the cone is between 5.8 mm and 6.4 mm, preferably between 6.0 mm and 6.2 mm, particularly preferably 6.1 mm. Through this design of male insertion element deviations in the fit of the corresponding female receptacle section can be compensated as a sealing area is formed by the area of the cone with the greater slope angle 168 and the external diameter at the end of the area with the greater slope angle 168.

In a further example of embodiment shown in FIGS. 4 and 5 the lockable connection system in accordance with the invention is used on at least one connection point of a Y connector. This ensures that all or certain connection points can only be connected to certain counter sections. For example, a connection 41, 51 of the Y connector has a female connection element in accordance with the invention with internal cones in accordance with the invention with corresponding fixing sections. The external fixing section in this form of embodiment is characterised in that it has two radially outwardly protruding lobes 43, 43′, 53, 53′.

Forms of embodiment with male connection element in accordance with the invention with corresponding fixing sections, well as connection elements on the other connection points are also of course part of the present invention. By way of the example of embodiment described above it can be ensured that, for example, only media containers appropriate for the application, such as for artificial feeding, can be connected to the Y connector. According the connection system in accordance with the invention allow the tight fitting and unambiguous, i.e. confusion-free, application-defined connection of different components.

In a further example of embodiment shown in FIGS. 6 and 7 the lockable connection system in accordance with the invention is used on a closing cap. This ensures that certain connecting points are only connected/closed with certain closing caps. The outer fixing section 60, 70 is designed as union nut and has an internal thread 71 and on the outer side structural elements 63 for improved grip. Arranged centrically in the outer fixing section designed as a union nut is the male tube or cone-shaped insertion element 62, 72 designed as a cone. When using the closing cap the male, tube-shaped insertion element engages in a female counterpiece, which has a corresponding receptacle section, of a corresponding connection system. The female counterpiece also has a fixing section, which corresponds with the fixing section of the closing cap in accordance with the invention designed as a union nut, and thus has a corresponding external thread.

In a further example of embodiment shown in FIGS. 8 and 9 the lockable connection system in accordance with the invention is used on a closing cap. The outer fixing section 80, 90 is designed as a union nut, and correspondingly has an internal thread 91 and on the outside structural elements 83 for better grip. Arranged centrically in the outer fixing section designed as a union nut is the male tube or cone-shaped insertion element 82, designed as a cone. The closing cap can more particularly be connected by means of a lug connected to the closing cap to the connection partner in order to hold the closing cap on the relevant connection partner even when in the open state. This lug is connected to the closing cap by way of a further lug, whereby closing cap has a receptacle area 84, 94 for the corresponding lug. In this case the two lugs are connected to an at least partially elastic elongated area.

In a further example of embodiment shown in FIGS. 10 and 11 the lockable connection system in accordance with the invention is used on a closing cap. The outer fixing section 100, 110 is designed as a union nut, and correspondingly has an internal thread 111 and on the outside structural elements 103 for better grip. Arranged centrically in the outer fixing section designed as a union nut is the male tube or cone-shaped insertion element 102, 112 designed as a cone. The male, cone-shaped insertion element 112 is designed in accordance with the invention so that longitudinally it is completely surrounded by the surrounding outer fixing section; it does not therefore project from the fixing section designed as a union nut 100, 110. This effectively prevents the insertion element 112 coming into contact with unsterile material during assembly of the closing cap and thereby becoming contaminated and bringing germs into the fluid system. When using the closing cap the male tube or cone-shaped insertion element engages in a female counterpiece, which has a corresponding receptacle section, of a corresponding connection system. The female counterpiece also has a fixing section, which corresponds with the fixing section of the closing cap in accordance with the invention designed as a union nut, and thus has a corresponding external thread.

In a further example of embodiment shown in FIGS. 12 and 13 the lockable connection system in accordance with the invention is used on a closing cap. The outer fixing section 120, 130 is designed as a union nut, and correspondingly has an internal thread 131 and on the outside structural elements 123 for better grip. Arranged centrically in the outer fixing section designed as a union nut is the male tube or cone-shaped insertion element 122, 132 designed as a cone. The male, cone-shaped insertion element 132 is designed in accordance with the invention so that longitudinally it is completely surrounded by the surrounding outer fixing section; it does not therefore project from the fixing section designed as a union nut 120, 130. The closing cap can more particularly be connected by means of a lug connected to the closing cap to the connection partner in order to hold the closing cap on the relevant connection partner even in the open state. This lug is connected to the closing cap by way of a further lug, whereby closing cap has a receptacle area 124, 134 for the corresponding lug. In this case the two lugs are connected to an at least partially elastic elongated area.

In a further example of embodiment shown in FIGS. 14 and 15 the lockable connection system in accordance with the invention is used on a closing cap. The outer fixing section 140, 150 is designed as a union nut, and correspondingly has an internal thread 151 and on the outside structural elements 143 for better grip. Arranged centrically in the outer fixing section designed as a union nut is the male tube or cone-shaped insertion element 152 designed as a cone. The outer fixing section 140, 150 is elongated and has thread-free sleeve-shaped extension 155. This serves on the one hand to protect the male insertion element 152 and on the other hand as assembly guide.

In summary, through aligned variation of the two element of the connection system, secure, confusion-free connection of various components is assured. Thus, through selecting appropriate components an entire system fulfilling the relevant requirements can be provided. An example of this is in the field of artificial feeding. Here, due to the high viscosity of the media to be conveyed there are special requirement with regard to the size of the throughput opening and the tubing system as a whole, but also in terms of the pressure-resistance of the individual components of the system, such as, for example, in the use of multi-way valve.

1. A lockable connection system for components that have fluids flowing there through for medicine and medical technology comprising:
a female, tube or cone-shaped receptacle, which has an internal receptacle section and an external first fixing section and,
a male, tube or cone-shaped insertion element for accommodating in the female receptacle section and an external second fixing section, which interacts with the first fixing section to fix the connection system, wherein
the first or second fixing section has at least two radially outwardly protruding lobes each having a radial outer edge which lies on a diameter greater than 7.83 mm and/or the at least two radially outwardly protruding lobes each has a base and an axial thickness of which on the base is greater than 1.3 mm.

2. The lockable connection system in accordance with claim 1, wherein the outer edges lie on a diameter which is greater than 7.83 mm and less than 20 mm and/or the axial thicknesses are greater than 1.3 mm and less than 5 mm.

3. The lockable connection system of claim 1 wherein
the first or second fixing section has at least three radially outwardly protruding lobes.

4. The lockable connection system in accordance with claim 1, wherein
the second or first fixing section has a sleeve for accommodating the lobes which has an internal thread.

5. The lockable connection system in accordance with claim 4, wherein the sleeve can be rotatably moved independently of the male insertion element or the female receptacle section.

6. The lockable connection system in accordance with claim 4 wherein a number of threads corresponds with a number of lobes.

7. The lockable connection system in accordance with claim 1, wherein the male insertion element has a cone with two different slope angles (α, β), whereby a first slope (α) of the cone in an area towards a base of insertion element is greater than a second slope (β) of the cone in an end area of the insertion element.

8. The lockable connection system in accordance with claim 7, wherein the male insertion element at an end towards a base of a section with the greater slope has a diameter of 5.8 mm to 6.4 mm.

9. The lockable connection system in accordance with claim 1, wherein the components through which fluid flows are transfusion tubes, multi-way valves, multiple distributors, injection equipment, needles, accesses, or combinations thereof.

10. The lockable connection system in accordance with claim 1, wherein surfaces of the components through which fluid flows that come into contact with the fluid have an antiseptic and/or antimicrobial material at least in parts.

11. The lockable connection system in accordance with claim 10, wherein the antiseptic or antimicrobial material has at least one germ-killing material which is selected from a group which includes highly porous silver, ion-free silver, silver compounds, microsilver, compounds releasing metal ions, and combinations thereof.

12. The lockable connection system in accordance with claim 10, wherein at least the surfaces coming into contact with the fluid are at least in part made of an amorphic copolyester.

13. The lockable connection system in accordance with claim 12, wherein in addition to the copolyester, other materials are used which are selected from a group containing duroplastic and thermoplastic synthetic materials, polylphenyl sulphide, polypropylene, poly-1-butene, polyvinylchloride, polyvinylidene chloride, polymethyl metaacrylate, polyacryl nitrile, polystyrene, polysulphone, polyacetal, polyvinyl alcohol, polyvinyl acetate, ionomers, fluoroplastics, polyethylene, polyamide, more particularly a partial aromatic polyamide, polycarbonate, polyester, copolyester, polyphenyl oxide, polysulphone, polyvinyl acetate, polyurethane and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, polyvinyl chloride, unsaturated polyester resin, antimicrobial or antiseptic materials, highly porous silver, ion-free silver, silver compounds, micro-silver, metal ion-releasing compounds, and combinations thereof.

14. The lockable connection system in accordance with claim 13, wherein the materials contains no softening agents.

15. A tube or cone-shaped receptacle or adapted to receive an insertion element to form a lockable connection system, comprising:
either the tube or cone-shaped receptacle or the insertion element includes an external fixing section which has at least two radially outwardly protruding lobes having a radial outer edge and the radial outer edges lie on a diameter greater than 7.83 mm and/or the at least two radially outwardly protruding lobes have an axial thickness of which on a base is greater than 1.3 mm.

16. A tube or cone-shaped receptacle or adapted to be inserted into an insertion element to form a lockable connection system, comprising:
either the tube or cone-shaped receptacle or the insertion element includes an external fixing section with at least three radially outwardly protruding lobes.

17.-31. (canceled)
